(19) United States
(12) Patent Application Publication
(10) Pub. No.: US 2011/0184520 A1
(43) Pub. Date: Jul. 28, 2011

(54) SACRO-ILIAC JOINT IMPLANT, METHOD AND APPARATUS

(75) Inventor: Hai H. Trieu, Memphis, TN (US)

(73) Assignee: WARSAW ORTHOPEDIC, INC., Warsaw, IN (US)

(21) Appl. No.: 12/694,312

(22) Filed: Jan. 27, 2010

(51) Int. Cl. A61F 2/44

(52) U.S. Cl. 623/17.11

(57) ABSTRACT

A sacro-iliac implant system includes at least one implant including a body defining an outer surface and being expandable in at least one dimension. The body is configured to expand in the at least one dimension from a first orientation to a second orientation such that the outer surface engages and spaces apart opposing articular surfaces of a sacro-iliac joint to fix the body with the articular surfaces. Methods of use are disclosed.

### TECHNICAL FIELD

The present disclosure generally relates to medical devices for the treatment of musculoskeletal disorders, and more particularly to an implant system and method for treating the sacro-iliac joint.

### BACKGROUND

The sacro-iliac (“SI”) joint is a diarthrodial joint that joins the sacrum to the ilium bones of the pelvis. In the SI joint, the sacral surface has hyaline cartilage that moves against fibrocartilage of the iliac surface. The spinal column is configured so that the weight of an upper body rests on the SI joints at the juncture of the sacrum and ilia. Stress placed on the SI joints in an upright position of the body makes the lower back susceptible to injury.

Disorders of the SI joint can cause low back and radiating buttock and leg pain in patients suffering from degeneration and laxity of the SI joint. In some cases, the SI joint can undergo dehydration and destabilization, similar to other cartilaginous joints, which causes significant pain. The SI joint is also susceptible to trauma and degeneration, from fracture and instability. It is estimated that disorders of the SI joint are a source of pain for millions of people suffering from back and radicular symptoms.

Non-surgical treatments, such as medication, injection, mobilization, rehabilitation and exercise can be effective, however, may fail to relieve the symptoms associated with these disorders. Surgical treatment of these disorders includes stabilization and/or arthrodesis. Stabilization can include the use of bone screws that are directly threaded into bone. Arthrodesis may include immobilization of a joint. The present disclosure describes an improvement over these prior art technologies.

### SUMMARY OF THE INVENTION

Accordingly, an implant system and method is provided for treating the SI joint. It is contemplated that the system may include an implant configured for disposal with the SI joint. It is further contemplated that the implant system and method may be employed for an arthrodesis treatment.

In one particular embodiment, in accordance with the principles of the present disclosure, a sacro-iliac implant system is provided. The sacro-iliac implant system includes at least one implant including a body defining an outer surface and being expandable in at least one dimension. The body is configured to expand in the at least one dimension from a first orientation to a second orientation such that the outer surface engages and spaces apart opposing articular surfaces of a sacro-iliac joint to fix the body with the articular surfaces.

In one embodiment, the sacro-iliac implant system includes at least one implant including an elongated, tubular body defining an outer surface and being radially expandable. The body is configured to expand from a first, collapsed orientation to a second inflated orientation such that the outer surface engages and spaces apart opposing articular surfaces of a sacro-iliac joint to fix the body with the articular surfaces. A delivery instrument includes a distal portion and a proximal portion. The distal portion includes an inflatable member detachably connected with the implant body and the proximal portion including an inflator.

In one embodiment, a method for treating a sacro-iliac joint is provided. The method includes the steps of: providing at least one implant including a body defining an outer surface and being expandable in at least one dimension, the body being configured to expand in the at least one dimension from a first orientation to a second orientation such that the outer surface engages and spaces apart opposing articular surfaces of a sacro-iliac joint to fix the body with the articular surfaces; delivering the implant to the sacro-iliac joint between the opposing articular surfaces; disposing the body within the sacro-iliac joint in a first, non-expanded orientation; providing an expanding device; expanding the body to a second, expanded orientation such that the outer surface engages and spaces apart the opposing articular surfaces with the expanding device; and removing the expanding device from the sacro-iliac joint.

### BRIEF DESCRIPTION OF THE DRAWINGS

The present disclosure will become more readily apparent from the specific description accompanied by the following drawings, in which:

FIG. 1 is a plan view, in part cross section, of one particular embodiment of an implant system in accordance with the principles of the present disclosure and a sacro-iliac/ilio-pelvic region;

FIG. 2 is a side view of the implant system, partially shown in phantom, and the region shown in FIG. 1;

FIG. 2A is a side cutaway view, in part cross section, of the implant system shown in FIG. 1;

FIG. 3 is a plan view, in part cross section, of the implant system and the region shown in FIG. 1;

FIG. 4 is a side view of the implant system, partially shown in phantom, and the region shown in FIG. 3;

FIG. 4A is a side cutaway view, in part cross section, of the implant system shown in FIG. 3;

FIGS. 5 A-B are perspective views of one embodiment of an implant of the implant system shown in FIG. 1;

FIGS. 6 A-B are perspective views of one embodiment of an implant of the implant system shown in FIG. 1;

FIGS. 7 A-B are perspective views of one embodiment of an implant of the implant system shown in FIG. 1;

FIGS. 8 A-B are perspective views of one embodiment of an implant of the implant system shown in FIG. 1;

FIGS. 9 A-G are cross section views of embodiments of an implant of the implant system shown in FIG. 1;

FIG. 10 is a side view of one embodiment of an expanding device of the implant system shown in FIG. 1; and

FIGS. 11A-D are side views of embodiments of an implant of the implant system shown in FIG. 1.

Like reference numerals indicate similar parts throughout the figures.

### DETAILED DESCRIPTION OF THE INVENTION

The exemplary embodiments of the implant system and methods of use disclosed are discussed in terms of medical devices for treating the SI joint. It is envisioned that the implant system and methods of use disclosed provide stability and maintains structural integrity while reducing stress on the SI joint. It is further envisioned that the present disclosure may be employed to treat musculoskeletal disorders including sacro-Iliac dysfunction or syndrome, dehydration, destabilization, laxity, fracture, tumor, spinal disorders and other orthopedic disorders. It is contemplated that the present disclosure may be employed with surgical treatments, including open surgery, percutaneous and minimally invasive procedures of such disorders, such as, for example, arthrodesis including fusion, bone graft and implantable prosthetics. It is further contemplated that the present disclosure may be employed with other osteal and bone related applications, including those associated with diagnostics and therapeutics. The disclosed implant system and methods may be employed in a surgical treatment with a patient in a prone or supine position, employing a posterior, lateral, inferior, posterior-inferior, superior or anterior approach. The present disclosure may be employed with procedures for treating the lumbar, cervical, thoracic and pelvic regions of a spinal column.

The present invention may be understood more readily by reference to the following detailed description of the invention taken in connection with the accompanying drawing figures, which form a part of this disclosure. It is to be understood that this invention is not limited to the specific devices, methods, conditions or parameters described and/or shown herein, and that the terminology used herein is for the purpose of describing particular embodiments by way of example only and is not intended to be limiting of the claimed invention. Also, as used in the specification and including the appended claims, the singular forms “a,” “an,” and “the” include the plural, and reference to a particular numerical value includes at least that particular value, unless the context clearly dictates otherwise. Ranges may be expressed herein as from “about” or “approximately” one particular value and/or to “about” or “approximately” another particular value. When such a range is expressed, another embodiment includes from the one particular value and/or to the other particular value. Similarly, when values are expressed as approximations, by use of the antecedent “about,” it will be understood that the particular value forms another embodiment. It is also understood that all spatial references, such as, for example, horizontal, vertical, top, upper, lower, bottom, left and right, are for illustrative purposes only and can be varied within the scope of the disclosure. For example, the references “upper” and “lower” are relative and used only in the context to the other, and are not necessarily “superior” and “inferior”.

The following discussion includes a description of an implant system, related components and exemplary methods of employing the implant system in accordance with the principles of the present disclosure. Alternate embodiments are also disclosed. Reference will now be made in detail to the exemplary embodiments of the present disclosure, which are illustrated in the accompanying figures.

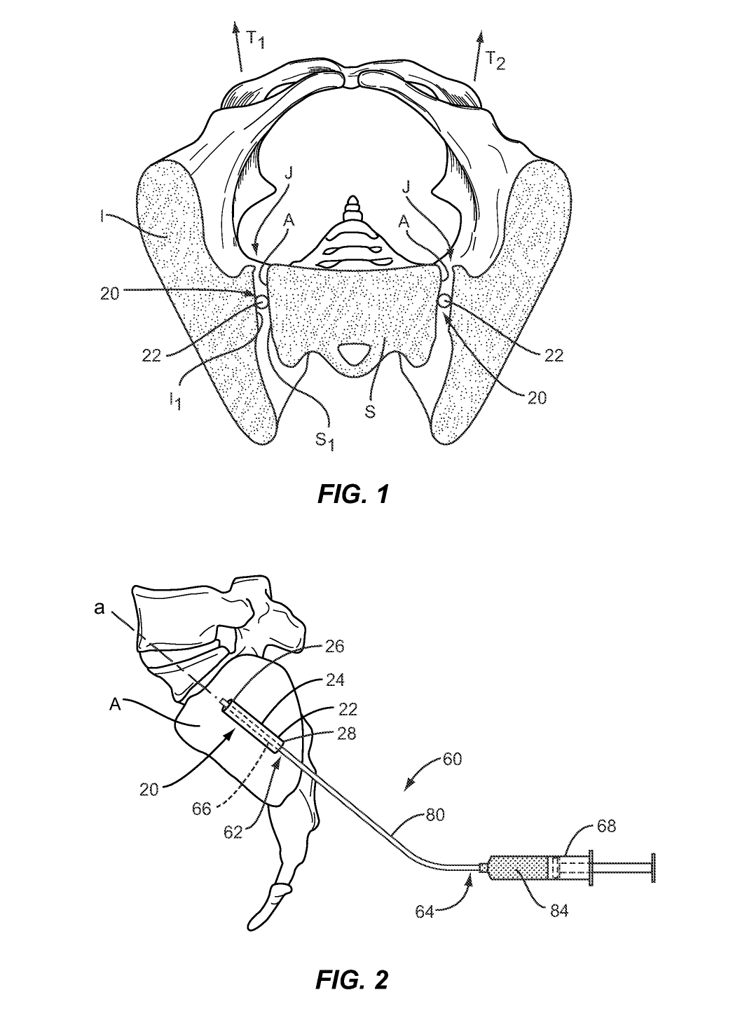

The following discussion includes a description of an implant system, related components and exemplary methods of employing the implant system in accordance with the principles of the present disclosure. Alternate embodiments are also disclosed. Reference will now be made in detail to the exemplary embodiments of the present disclosure, which are illustrated in the accompanying figures. Turning now to FIGS. 1-4A, there are illustrated components of the implant system in accordance with the principles of the present disclosure.

The components of the implant system are fabricated from materials suitable for medical applications, including metals, synthetic polymers, ceramics, bone, biocompatible materials and/or their composites, depending on the particular application and/or preference of a medical practitioner. For example, components of the implant system, such as, for example, an implant body, an outer surface of the implant body and/or portions thereof, cavities of the implant body, which may be monolithically formed, integrally connected or configured as an insert with the body, fastening elements and/or instruments and/or expanding devices, discussed below, can be fabricated from materials such as commercially pure titanium, titanium alloys, Grade 5 titanium, super-elastic titanium alloys, cobalt-chrome alloys, stainless steel alloys, superelastic metallic alloys (e.g. Nitinol, super elasto-plastic metals, such as GUM METAL® manufactured by Toyota Material Incorporated of Japan), thermoplastics such as polyaryletherketone (PAEK) including polyetheretherketone (PEEK), polyetherketoneketone (PEKK) and polyetherketone (PEK), carbon fiber reinforced PEEK composites, PEEK-BaSO₄ composites, ceramics and composites thereof such as calcium phosphate (e.g. SKELITE™ manufactured by Biologix Inc.), rigid polymers including polyphenylene, polyamide, polyimide, polyetherimide, polyethylene, polyurethanes of any durometer, epoxy, silicone, bone material including autograft, allograft, xenograft or transgenic cortical and/or corticocancellous bone, and tissue growth or differentiation factors. Different components of the implant system may have alternative material composites to achieve various desired characteristics such as strength, rigidity, elasticity, compliance, biomechanical performance, durability and radiolucency or imaging preference. The components of the implant system may also be fabricated from a heterogeneous material such as a combination of two or more of the above-described materials.

It is envisioned that the components of the implant system can be manufactured via various methods. For example, the implant body can be manufactured and assembled via injection-molding, insert-molding, overmolding, compression molding, transfer molding, co-extrusion, pultrusion, dip-coating, spray-coating, powder-coating, porous-coating, milling from a solid stock material, and their combinations. One skilled in the art, however, will realize that such materials and fabrication methods suitable for assembly and manufacture, in accordance with the present disclosure, would be appropriate.

The implant system includes an orthopedic implant, such as, for example, a sacro-iliac implant 20, which is configured, for example, to treat S-I joint disorders including those caused by degeneration or trauma. It is contemplated that sacro-iliac implant 20 may be employed for arthrodesis applications, as will be described.

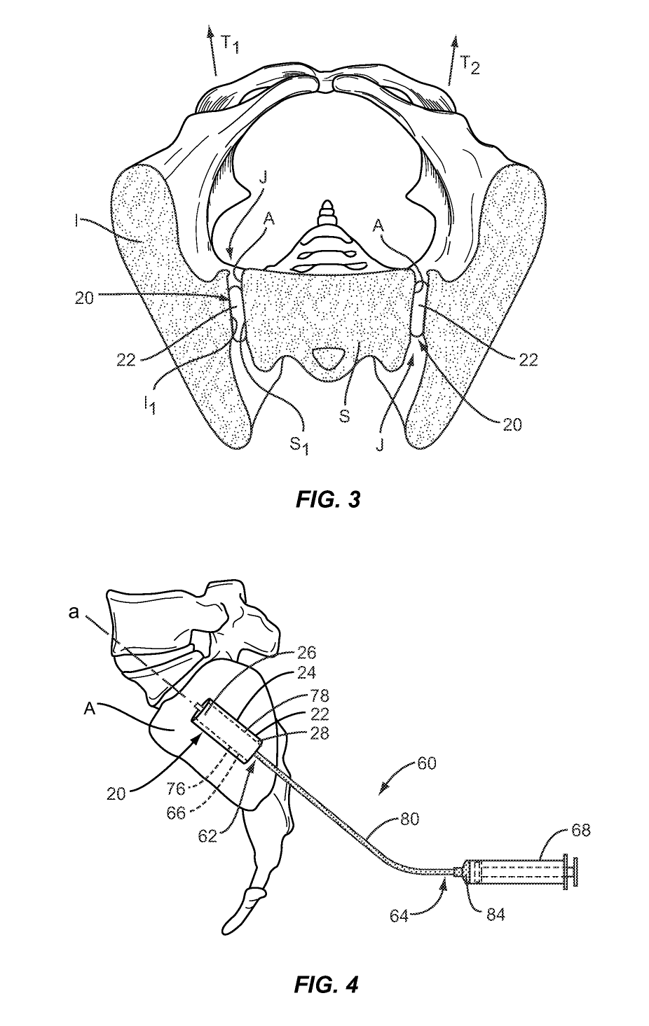

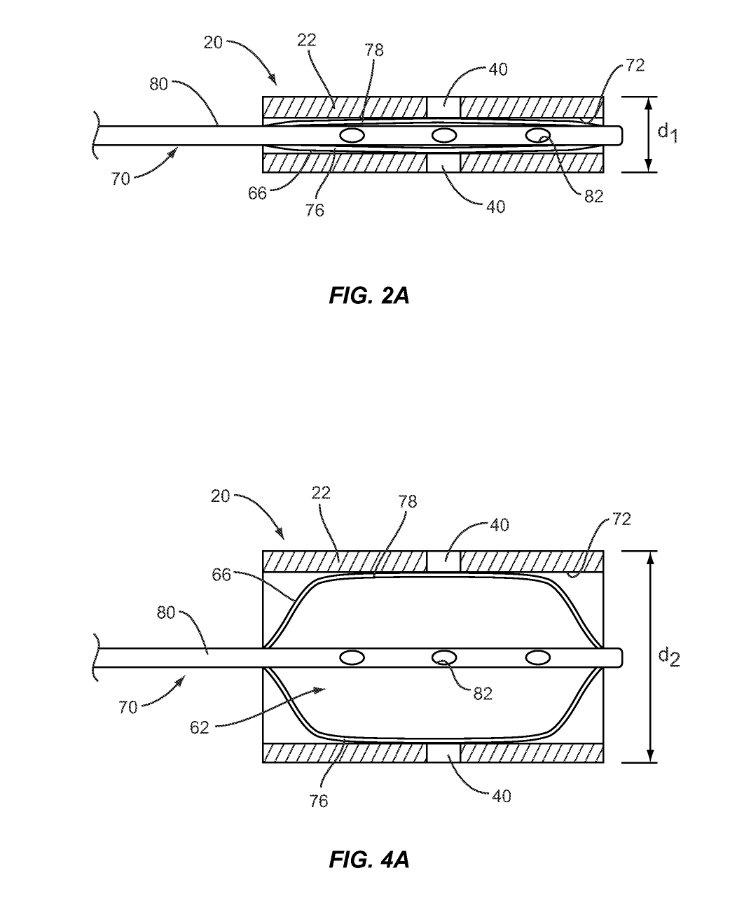

Sacro-iliac implant 20 includes a body 22 that is elongated along a longitudinal axis a thereof. Body 22 has a tubular configuration and defines an outer surface 24. Body 22 is expandable and configured for radial expansion. Body 22 is configured to expand from a first, collapsed orientation (FIGS. 1, 2 and 2A) to a second inflated orientation (FIGS. 3, 4 and 4A) such that outer surface 24 engages and spaces apart opposing articular surfaces A of a sacro-iliac joint J to fix body 22 with articular surfaces A. It is contemplated that body 22 may be elastically deformable or plastically deformable.

It is envisioned that body 22 is initially disposed in a relatively smaller cross-sectional dimension, for example, diameter, height and/or width, which may include a first orientation such as, for example, a collapsed or pre-deployed orientation. It is further envisioned that body 22 can be expanded or deployed in situ such that a cross-sectional dimension of body 22 is increased and outer surface 24 is secured within the SI joint. It is contemplated that body 22 is expandable in one or a plurality of dimensions, such as, for example, height, width, length, diameter, radial direction and/or volumetric direction. In one embodiment, expansion of implant 20 is reversible for removal and/or revision. It is envisioned that body 22 is expandable via a mechanical linkage such as, for example, a mechanical jack or, alternatively, with a high-pressure non-compliant balloon as will be described.

It is contemplated that articular surface A may refer to a sacral surface S₁ of a sacrum S and/or an iliac surface I₁ of an ilium I. Body 22 is configured to engage opposing articular surfaces such as sacral surface S₁ and iliac surface I₁ and/or opposing valleys or peaks of an individual sacrum S or ilium I. Body 22 may have a hollow, porous or cage configuration. Outer surface 24 has a continuously even or smooth configuration. It is contemplated that outer surface 24 is configured to substantially match articular surface(s) A and/or may be substantially smooth, rough, textured, spiked, porous, semi-porous, dimpled, keeled and/or polished. It is further contemplated that the volumetric dimension of body 22 may include solid, partially solid, porous and/or semi-porous.

Body 22 extends from a first end 26 to a second end 28. Body 22 has a first diameter d₁ (FIG. 2A) in the first non-expanded orientation and a second diameter d₂ (FIG. 4A) in the expanded orientation, according to the requirements of the particular application. It is envisioned that diameter d₁ may be in a range of approximately 1-10 millimeters (mm). It is further envisioned that diameter d₁ may be varied depending on whether an SI joint is drilled and/or tapped before insertion of an implant. It is contemplated that diameter d₂ may be in a range of approximately 5-40 mm. It is further contemplated that diameter d₂ may be varied depending on the cross section of body 22. For example, in one embodiment, body 22 has an elliptical cross section such that diameter d₁ is in a range of approximately 5-10 mm and diameter d₂ is in a range of approximately 10-40 mm.

This configuration of body 22 facilitates disposal and fixation of body 22 within a SI joint, such that, for example, body 22 separates articular surfaces A to dilate the SI joint and prevent joint surfaces from undesired engagement such as that caused by degeneration and cartilage wear. It is contemplated that such spacing apart of the articular surfaces of the SI joint tensions ligaments, supports the SI joint and provides maximum stabilization of the SI joint. It is further contemplated that the overall and/or cross-sectional geometry of body 22 may have various configurations, for example, round, oval, oblong, triangular, rectangular, polygonal, irregular, uniform, non-uniform, consistent or variable.

It is envisioned that body 22 can be variously configured and dimensioned with regard to size, shape, thickness, geometry and material. Body 22 may also be formed of one or a plurality of elements such as spaced apart portions, staggered patterns and mesh. It is envisioned that the particular geometry and material parameters of body 22 may be selected to modulate the flexibility or stiffness of sacro-iliac implant 20, such as those examples discussed herein. For example, body 22 can be configured to have varying ranges or degrees of flexibility or stiffness such as rigid, compliant, or reinforced. Depending on the flexibility or stiffness of body 22, the flexibility or stiffness of sacro-iliac implant 20 can be contoured according to the requirements of a particular application. It is contemplated that the ability to vary stiffness of sacro-iliac implant 20 promotes fusion of the elements of sacro-iliac joint J. It is envisioned that the components of sacro-iliac implant 20 may be monolithically formed, integrally connected or arranged with attaching elements.

In one embodiment, body 22 defines a plurality of cavities, such as, for example, openings 40 extending through body 22, as shown in FIGS. 2A and 4A. Openings 40 are disposed with body 22 in an orientation facing articular surfaces A, sacral surface S₁ and iliac surface I₁. It is envisioned that body 22 may include one or a plurality of openings 40. It is further envisioned that the cavities may include through holes, slots, voids, indentations, and/or non-interference configurations and dimensions. In one embodiment, openings 40 are configured for bony ingrowth in a fusion application.

In one embodiment, one or a plurality of openings 40 may be configured to expel and/or elute at least one agent therefrom. Such an opening(s) 40 may includes one or a plurality of agent reservoirs. The agent reservoirs can be configured as drug depots with medication for pain and may include antibiotics and/or therapeutics. Diffusion of such agents can occur through openings 40. It is envisioned that body 22 includes a network of agent diffusing openings 40 (not shown) interconnected and/or in fluid communication. It is contemplated that body 22 and/or each of openings 40 may include one or a plurality of agents. Openings 40 may be oriented parallel to articular surfaces of a SI joint, perpendicular, randomly oriented, and/or configured for multiple directional expulsion or eluting.

It is envisioned that the agent reservoirs contains active agents and may include one or a plurality of therapeutic agents and/or pharmacological agents for release, including sustained release, into SI joint J to treat, for example, pain, inflammation and degeneration. The agents may include pharmacological agents, such as, for example, antibiotics, pain medications, analgesics, anesthetics, anti-inflammatory drugs including but not limited to steroids, anti-viral and anti-retroviral compounds, therapeutic proteins or peptides, therapeutic nucleic acids (as naked plasmid or a component of an integrating or non-integrating gene therapy vector system), and combinations thereof.

The agent may also include analgesics or anesthetics such as acetic acid derivatives, clonidine, COX-2 selective inhibitors, COX-2 inhibitors, enolic acid derivatives, propionic acid derivatives, salicylic acid derivatives, opioids, opioid/nonopioid combination products, adjuvant analgesics, and general and regional/local anesthetics.

The agent may also include antibiotics such as, for example, amoxicillin, beta-lactamases, aminoglycosides, beta-lactam (glycopeptide), clindamycin, chloramphenicol, cephalosporins, ciprofloxacin, erythromycin, fluoroquinolones, macrolides, metronidazole, penicillins, quinolones, rapamycin, rifampin, streptomycin, sulfonamide, tetracyclines, trimethoprim, trimethoprim-sulfamethoxazole, and vancomycin.

The agent may also include immunosuppressives agents, such as, for example, steroids, cyclosporine, cyclosporine analogs, cyclophosphamide, methylprednisone, prednisone, azathioprine, FK-506, 15-deoxyspergualin, prednisolone, methotrexate, thalidomide, methoxsalen, rapamycin, leflunomide, mizoribine (Bredinin™), brequinar, deoxyspergualin, and azaspirane (SKF 105685), Orthoclone OKT™ 3 (muromonab-CD3). Sandimmune™, Neoral™, Sangdya™ (cyclosporine), Prograf™ (FK506, tacrolimus), Cellcept™ (mycophenolate motefil, of which the active metabolite is mycophenolic acid), Imuran™ (azathioprine), glucocorticosteroids, adrenocortical steroids such as Deltasone™ (prednisone) and Hydeltrasol™ (prednisolone), Folex™ and Mexate™ (methotrxate), Oxsoralen-Ultra™ (methoxsalen) and Rapamuen™ (sirolimus).

It is envisioned that openings 40 are capable of accepting at least one agent before, during and/or after implantation with joint J, holding the at least one agent in a reservoir, and/or delivery in vivo of the at least one agent to tissues of joint J and tissues surrounding joint J, including bone. Openings may be replenished, via one or a plurality of iterations, with therapeutic and/or pharmacological agents.

In one embodiment, the implant system includes a plurality of bodies 22, described above. It is contemplated that employing the plurality of bodies 22 can optimize the amount sacro-iliac joint J can be spaced apart such that a joint space dimension can be preselected. The plurality of bodies 22 can be inserted through the same or an alternate trajectory. The plurality of bodies 22 can be oriented in a side by side engagement, spaced apart and/or staggered. It is envisioned that one or all of the plurality of bodies 22 may be inserted via a trajectory oriented from an anterior, posterior, superior or inferior direction, similar to that described herein. It is further envisioned that one or a plurality of bodies 22 may be used.

In assembly, operation and use, the implant system including sacro-iliac implant 20 is employed with a surgical procedure for treatment of a sacro-iliac joint J of a patient, as discussed herein. The implant system may also be employed with other surgical procedures. In particular, the implant system is employed with a surgical arthrodesis procedure, such as, for example, fusion for treatment of an applicable condition or injury of an affected sacro-iliac joint J, as shown in FIGS. 1-4A. It is contemplated that the implant system is inserted with sacro-iliac joint J to space apart articular joint surfaces, establish joint tension, provide support and maximize stabilization of sacro-iliac joint J. It is further contemplated that the implant system is inserted with a sacro-iliac joint J as a SI joint spacer to restore ligamentous tension, eliminate painful micro-motion, and/or separate and cushion opposing articulating surfaces that cause pain. It is envisioned that the implant system may maintain joint tension without promoting bone growth.

In use, to treat the affected section of sacro-iliac joint J, a medical practitioner obtains access to a surgical site including sacro-iliac joint J in any appropriate manner, such as through incision and retraction of tissues. It is envisioned that the implant system may be used in any existing surgical method or technique including open surgery, mini-open surgery, minimally invasive surgery and percutaneous surgical implantation, whereby sacro-iliac joint J is accessed through a mini-incision, or sleeve that provides a protected passageway to the area. Once access to the surgical site is obtained, the particular surgical procedure is performed for treating the sacro-iliac joint disorder. The implant system is then employed to augment the surgical treatment. The implant system can be delivered or implanted as a pre-assembled device or can be assembled in situ. The implant system may be completely or partially revised, removed or replaced in situ. It is contemplated that one or all of the components of the implant system can be delivered to the surgical site via manual manipulation and/or a free hand technique.

First and second trajectories T₁, T₂, as shown in FIG. 1, are defined for insertion and/or injection of bodies 22 of sacro-iliac implants 20 within sacro-iliac joints J. Each implant 20 is inserted via the protected passageway along the defined trajectories T₁, T₂ into sacro-iliac joints J. Each cavity of the respective sacro-iliac joints J are prepared along the respective trajectory for disposal of sacro-iliac implants 20. A guide wire, needle and/or trocar may be employed to penetrate tissues and create a pathway through the body of a patient to the SI joint site for disposal of implants 20.

The protected passageway includes a dilator/delivery tube (not shown) configured to deliver sacro-iliac implant 20 directly to the joint space of sacro-iliac joint J. It is envisioned that the dilator/delivery tube may include an endoscope camera tip for viewing insertion trajectory. It is further envisioned that the delivery tube includes a sleeve that can be retracted for expansion of body 22.

Sacro-iliac implant 20 is delivered to the joint space of sacro-iliac joint J and manipulated such that outer surface 24 of body 22 engages opposing articular surfaces A, according to the contour of articular surfaces A. It is contemplated that body 22 may engage only one or a plurality of articular surfaces A. It is further contemplated that body 22 can be oriented for a preset, predetermined and/or guided to a predetermined orientation for disposal with articular surfaces A.

The implant system including sacro-iliac implant 20 is employed with a surgical arthrodesis procedure for treatment of sacro-iliac joint J of a patient using a delivery/expanding instrument 60, configured for example as a high pressure balloon catheter. Instrument 60 includes a distal portion 62 and a proximal portion 64. Distal portion 62 includes an expanding device, such as, for example, an inflatable member 66 detachably connected with implant body 22 and proximal portion 64 includes an injector 68 that fluidly communicates via connection 70 with inflatable member 66 for inflation thereof. Injector 68 is actuated to provide the pressure required to expel a fluid 84 from injector 68 into inflatable member 66 for inflation thereof. It is contemplated that instrument 60 may employ various injectors for delivery of a flowable material to the expanding device such as, for example, a syringe, caulk-gun, mechanical injector and/or power injector. It is further contemplated that instrument 60 may employ alternative pressure generating devices to inflate inflatable member 66 such as a pump.

Body 22 is mounted with inflatable member 66 and disposed within the joint space of sacro-iliac joint J, as discussed. Body 22 defines an interior cavity 72 for disposal of inflatable member 66 therein. As shown in FIGS. 2 and 2A, body 22 has first diameter d₁ and is disposed in the first, collapsed orientation, and inflatable member 66 is deflated.

Inflatable member 66 includes elastically deformable walls 76, 78 attached with a shaft 80 of connection 70 in a sealed engagement. Walls 76, 78 are movable away from one another from the first orientation, an unexpanded configuration of body 22, as shown in FIGS. 2 and 2A, to the second orientation, an expanded configuration of body 22, as shown in FIGS. 4 and 4A. In the unexpanded configuration, body 22 has diameter d₁. In the expanded configuration, body 22 has diameter d₂.

Shaft 80 includes a lumen through which a gas, fluid or other material or agent 84 can be supplied through openings 82 to enlarge or inflate inflatable member 66. Upon delivery of implant 20 to the surgical site, injector 68 is actuated via manual, mechanical and/or processor element to force fluid into inflatable member 66, which is thereby inflated to separate walls 76, 78 and dispose body 22 in the second, expanded orientation, as shown in FIGS. 4 and 4A.

It is contemplated that shaft 80 can be rigid, semi-rigid, or flexible. It is further contemplated that shaft 80 can include one or a plurality of lumens to facilitate inflation with a biocompatible fluid, such as, for example, air or saline. Other embodiments contemplate that shaft 80 includes multiple lumens to, for example, communicate with openings 40 of body 22 to deliver bone graft, bone growth material or other suitable bone filler material into the joint space of sacro-iliac joint J. It is contemplated that inflatable member 66 is collapsed prior to or simultaneously with expelling or eluting of bone filler material and other agents disclosed herein such that the dedicated lumen (not shown) of shaft 80 for delivering such bone filler material and other agents communicate with openings 40. Such bone filler material and other agents delivered into body 22 may overflow into openings 40.

It is contemplated that one or a plurality of expanding devices may be employed with instrument 60, which may be alternately configured and dimensioned. It is further contemplated that inflatable member can have various geometric shapes such as, for example, conical, frusto-conical, spherical, cubic, spherical, polygonal, ovoid, long conical, long spherical, rectangular, tapered, stepped, dog-bone shape, offset shapes and combinations thereof.

Inflatable member 66 can be fabricated from any suitable material capable of withstanding the pressure supplied to enlarge or inflate inflatable member 66 in situ, such as, for example, various polymeric materials, including polyethylene, terephthalates, polyolefins, polyurethanes, nylon, polyvinyl chloride, silicone or other suitable material. It is envisioned that inflatable member 66 can be reinforced with woven or non-woven textile materials.

It is envisioned that bone filler materials delivered through openings 40 to facilitate fusion and/or treat the sacro-iliac joint disorder may include any suitable osteogenic material or composition, including autograft, allograft, xenograft, demineralized bone, and synthetic and natural bone graft substitutes, such as bioceramics and polymers, and osteoinductive factors. The terms osteogenic material or osteogenic composition used herein broadly include any material that promotes bone growth or healing including autograft, allograft, xenograft, bone graft substitutes and natural, synthetic and recombinant proteins, hormones and the like. It is further envisioned that body 22 can be inflated or back-filled with osteoconductive or osteoinductive materials.

Autograft can be harvested from locations such as the iliac crest using drills, gouges, curettes, and trephines and other tools and methods which are well known to surgeons in this field. It is envisioned that autograft is harvested from the iliac crest with a minimally invasive donor surgery. Natural and synthetic graft substitutes which replace the structure or function of bone are also contemplated for the osteogenic composition.

In some embodiments, the osteogenic compositions used can comprise a therapeutically effective amount to stimulate or induce bone growth of a bone inductive or growth factor or protein in a pharmaceutically acceptable carrier. Osteoinductive factors that are recombinant human bone morphogenetic proteins (rhBMPs) are contemplated because they are readily available and do not contribute to the spread of infectious diseases. The bone morphogenetic protein can be a rhBMP-2, rhBMP-7 or heterodimers thereof. It is contemplated that carriers may be used such as, for example, calcium sulphates, polylactic acids, polyanhydrides, collagen, calcium phosphates, polymeric acrylic esters, and demineralized bone.

Inflatable member 66 is deflated and removed from body 22. Instrument 60 is removed from the surgical site. Sacro-iliac implant 20 remains disposed with sacro-iliac joint J for treating the sacro-iliac joint disorder. Body 22 is configured for compliant engagement with articular surfaces A. Outer surface 24 engages and spaces apart respective opposing articular surfaces, sacral surface S₁ and iliac surface I₁, and is secured within joint J to stabilize and immobilize portions of sacrum S and ilium I of sacro-iliac joint J.

In one embodiment, the implant system may include fastening elements, such as, for example, screws configured for fixation with articular surfaces A through and external to body 22. Such screws are employed to secure joint surfaces and provide complementary stabilization and immobilization to sacro-iliac joint J. Sacro-iliac implant 20 may include locking structure, such as, locking elements formed or attached with body 22, to facilitate fixation of implant 20 within the joint space of sacro-iliac joint J, examples of which being discussed below.

It is contemplated that the implant system including sacro-iliac implant 20 may be employed during a surgical fusion procedure for treatment of a condition or injury, such as, degeneration or fracture. Fixation of sacro-iliac implant 20 with articular surfaces A and/or other portions of sacro-iliac joint J can be facilitated by the resistance provided by the joint space and/or engagement with the outer articular structures. Sacro-iliac implant 20 may include locking structure to facilitate fixation with articular surface(s) A. It is envisioned that such locking structure may include fastening elements such as, for example, clips, hooks, adhesives and/or flanges, as will be described below. It is further envisioned that in joint fusion applications of sacro-iliac implant 20, body 22 includes voids, cavities and/or openings for including therapeutic polynucleotides or polypeptides and bone growth promoting material, such as those described herein, which can be packed or otherwise disposed therein.

For example, outer surface 24 and/or openings 40 may include at least one agent including biocompatible materials, such as, for example, biocompatible metals and/or rigid polymers, such as, titanium elements, metal powders of titanium or titanium compositions, sterile bone materials, such as allograft or xenograft materials, synthetic bone materials such as coral and calcium compositions, such as hydroxyapatite, calcium phosphate and calcium sulfite, biologically active agents, for example, biologically active agents coated onto the exterior of implant 20 and/or applied thereto for gradual release such as by blending in a bioresorbable polymer that releases the biologically active agent or agents in an appropriate time dependent fashion as the polymer degrades within the patient. Suitable biologically active agents include, for example, bone morphogenic protein (BMP) and cytokines.

It is contemplated that the implant system and any fastening elements and attachments may be coated with an osteoconductive material such as hydroxyapatite and/or osteoinductive agent such as a bone morphogenic protein for enhanced bony fixation to the treated area. Sacro-iliac implant 20 can be made of radiolucent materials such as polymers. Radiomarkers may be included for identification under x-ray, fluoroscopy, CT or other imaging techniques.

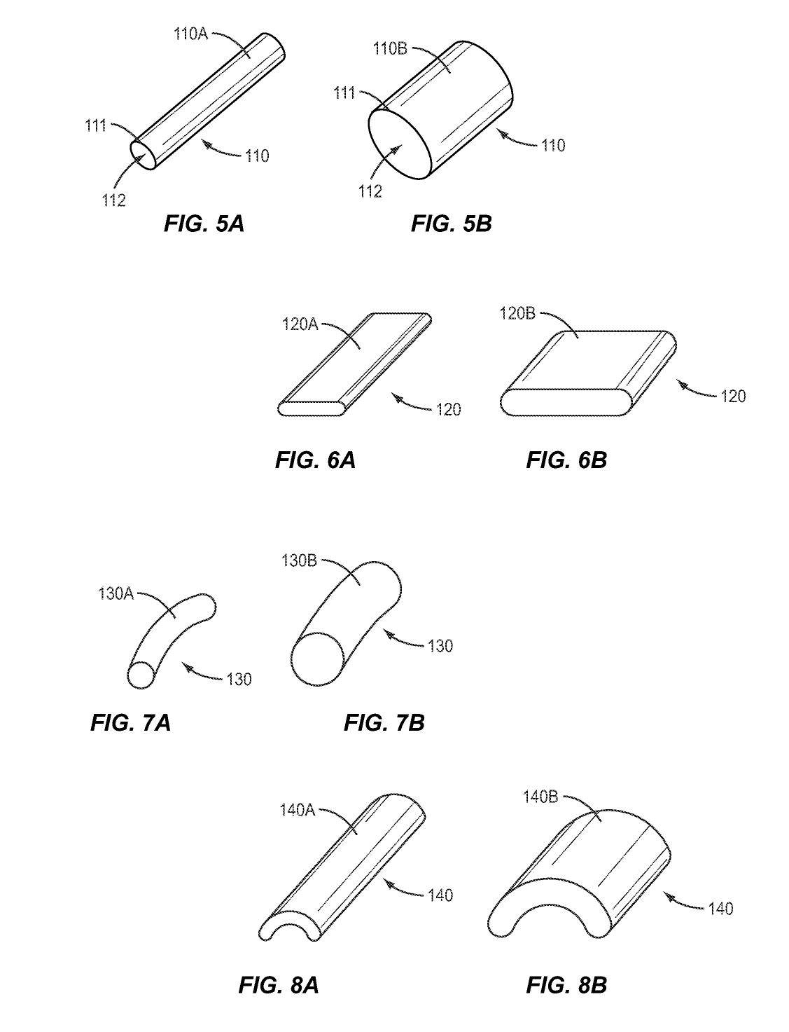

In one embodiment, as shown in FIGS. 5A and 5B, sacro-iliac implant 20 has a body 110, similar to body 22 described above. Body 110 has an unexpanded collapsed configuration 110A having a relatively small diameter and a radially expanded configuration 110B having a relatively larger diameter. Body 110 has an annular cross section defined by a cylindrical wall 111 surrounding an axial bore 112. The overall cross section of body 110 is substantially circular.

In one embodiment, as shown in FIGS. 6A and 6B, sacro-iliac implant 20 has a body 120, similar to body 22 described above. Body 120 has an unexpanded collapsed configuration 120A and an expanded configuration 120B. The cross section of expanded configuration 120B is elongated and relatively flat.

In one embodiment, as shown in FIGS. 7A and 7B, sacro-iliac implant 20 has a body 130, similar to body 22 described above. Body 130 has an unexpanded collapsed configuration 130A and an expanded configuration 130B.

In one embodiment, as shown in FIGS. 8A and 8B, sacro-iliac implant 20 has a body 140, similar to body 22 described above. Body 140 has an unexpanded collapsed configuration 140A and an expanded configuration 140B having an arcuate cross section.

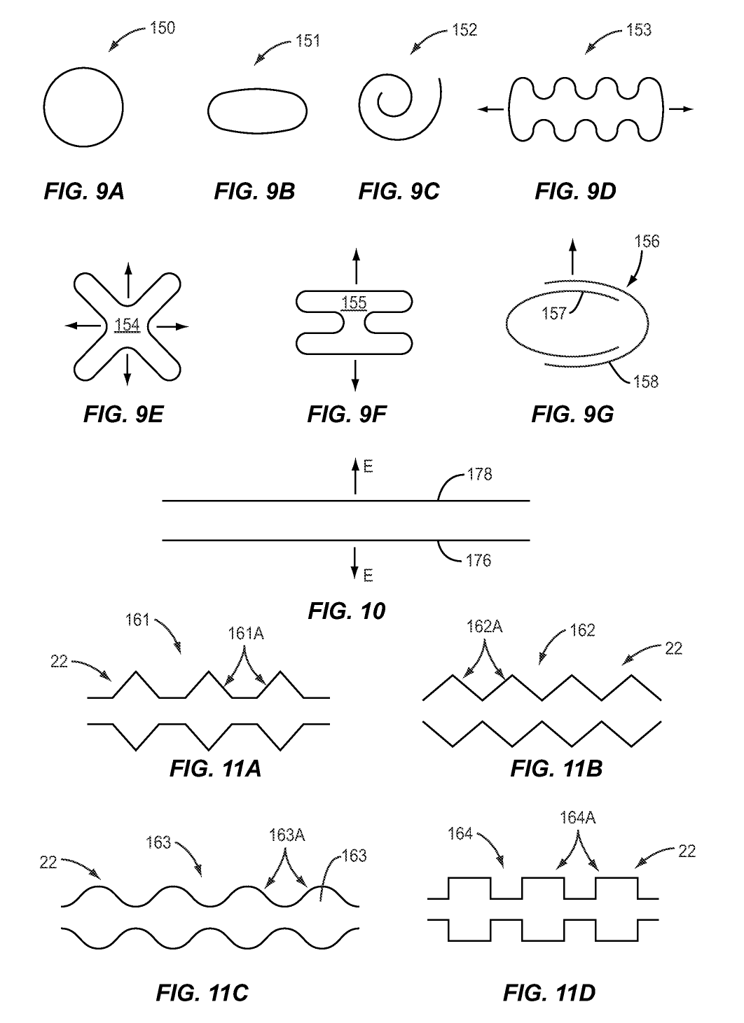

Referring to FIGS. 9A-9G, embodiments of sacro-iliac implant 20, similar to those described above, are shown in an unexpanded, collapsed orientation, in cross section. FIG. 9A shows a body 150 having a circular cross section. FIG. 9B shows a body 151 having a generally elliptical cross section. FIG. 9C shows a body 152 having a coiled configuration. FIG. 9D shows a body 153 having an accordion shaped cross section that expands laterally when expanded. FIG. 9E shows a body 154 having a generally cruciate configuration that expands outwardly. FIG. 9F shows a body 155 having an I-beam configuration that expands vertically. FIG. 9G shows a body 156 having interlocking portions, element 157 and element 158, which are engaged to form a generally U-shaped configuration. Body 156 expands vertically and laterally.

Referring to FIG. 10, in one embodiment similar to that described with regard to FIGS. 2A and 4A, the implant system is employed with and includes an instrument 160 for treating a sacro-iliac joint disorder of a sacro-iliac joint. Instrument 160 is a mechanical expanding device for expanding body 22 described above. Instrument 160 includes a first wall 176 and a second wall 178 that are configured to engage an inner wall surface of body 22 for expansion thereof. Walls 176, 178 are connected to mechanical linkages (not shown) to force walls 176, 178 in opposing directions, in the direction shown by arrows E, such that walls 176, 178 are movable away from one another from the first orientation, an unexpanded configuration of body 22, as shown in FIG. 2, to the second orientation, an expanded configuration of body 22, as shown in FIG. 4.

Referring to FIGS. 11A-11D, embodiments of sacro-iliac implant 20, similar to those described above, are shown in side view in an expanded, deployed orientation with outer surface 24 (FIGS. 2 and 4) including locking elements to facilitate fixation of body 22 with articular surface(s). FIG. 11A shows body 22 having an outer surface 161 including spaced apart serrations 161A. FIG. 11B shows body 22 having an outer surface 162 including continuous serrations 162A. FIG. 11C shows body 22 having an outer surface 163 including undulations 163A. FIG. 11D shows body 22 having an outer surface 164 including spaced apart rectangular ridges 164A.

It will be understood that various modifications may be made to the embodiments disclosed herein. Therefore, the above description should not be construed as limiting, but merely as exemplification of the various embodiments. Those skilled in the art will envision other modifications within the scope and spirit of the claims appended hereto.

What is claimed is:

1. A sacro-iliac implant system comprising:
at least one implant including a body defining an outer surface and being expandable in at least one dimension, the body being configured to expand in the at least one dimension from a first orientation to a second orientation such that the outer surface engages and spaces apart opposing articular surfaces of a sacro-iliac joint to fix the body with the articular surfaces.

2. A sacro-iliac implant system according to claim 1, wherein the body is elongated along a longitudinal axis thereof and has a tubular configuration.

3. A sacro-iliac implant system according to claim 1, wherein the at least one dimension includes radial expansion.

4. A sacro-iliac implant system according to claim 1, wherein the at least one dimension includes volumetric expansion.

5. A sacro-iliac implant system according to claim 1, further comprising a plurality of implant bodies.

6. A sacro-iliac implant system according to claim 1, wherein the implant body is configured for expansion via a mechanical linkage.

7. A sacro-iliac implant system according to claim 1, wherein the implant body is configured to be collapsible.

8. A sacro-iliac implant system according to claim 1, wherein the implant body is configured for removal from the sacro-iliac joint.

9. A sacro-iliac implant system according to claim 1, wherein the implant body is inflatable with an osteogenic material.

10. A sacro-iliac implant system according to claim 1, wherein the outer surface includes locking elements configured for engagement with the articular surfaces.

11. A sacro-iliac implant system according to claim 1, wherein the locking elements include serrations.

12. A sacro-iliac implant system according to claim 1, wherein the implant body is fabricated from a shape memory material.

13. A sacro-iliac implant system according to claim 1, wherein the implant body has a coiled cross sectional configuration.

14. A sacro-iliac implant system according to claim 1, wherein the implant body is fabricated from a material including at least one biologically active agent.

15. A sacro-iliac implant system according to claim 1, wherein the implant body is plastically deformable.

16. A sacro-iliac implant system comprising:
at least one implant including an elongated, tubular body defining an outer surface and being radially expandable, the body being configured to expand from a first, collapsed orientation to a second inflated orientation such that the outer surface engages and spaces apart opposing articular surfaces of a sacro-iliac joint to fix the body with the articular surfaces; and
a delivery instrument including a distal portion and a proximal portion, the distal portion including an inflatable member detachably connected with the implant body and the proximal portion including an inflator.

17. A sacro-iliac implant system according to claim 16, wherein the implant body is inflatable or back-filled with an osteogenic material.

18. A sacro-iliac implant system according to claim 16, wherein the outer surface includes locking elements configured for engagement with the articular surfaces.

19. A method for treating a sacro-iliac joint, the method comprising the steps of:
providing at least one implant including a body defining an outer surface and being expandable in at least one dimension, the body being configured to expand in the at least one dimension from a first orientation to a second orientation such that the outer surface engages and spaces apart opposing articular surfaces of a sacro-iliac joint to fix the body with the articular surfaces;
delivering the implant to the sacro-iliac joint between the opposing articular surfaces;
disposing the body within the sacro-iliac joint in a first, non-expanded orientation;
providing an expanding device;
expanding the body to a second, expanded orientation such that the outer surface engages and spaces apart the opposing articular surfaces with the expanding device; and
removing the expanding device from the sacro-iliac joint.

20. A method according to claim 19, wherein the step of expanding the body includes inflating or back-filling the body with an osteogenic material.
